(19) United States
(12) Patent Application Publication
(10) Pub. No.: US 2026/0368891 A1
(43) Pub. Date: Nov. 19, 2026

(54) INFORMATION PROCESSING DEVICE AND INFORMATION PROCESSING METHOD

(71) Applicant: CANON KABUSHIKI KAISHA, Tokyo (JP)

(72) Inventor: MASAMI KAWAGISHI, Kanagawa (JP)

(21) Appl. No.: 19/677,301

(22) Filed: May 14, 2026

(30) Foreign Application Priority Data
May 16, 2025 (JP) 2025-082589

(51) Int. Cl. G06T 7/00; A61B 6/00; A61B 6/03; G16H 50/20; A61B 6/50

(52) U.S. Cl. CPC G06T 7/0012; A61B 6/032; A61B 6/5217; G16H 50/20; A61B 6/50; G06T 2200/04; G06T 2207/10081; G06T 2207/20021; G06T 2207/30061

(57) ABSTRACT

An information processing device acquires medical image data; performs first inference about a predetermined disease to generate a plurality of first inference results corresponding to the plurality of first sub-regions by using a plurality of first sub-regions included in the medical image data as input; detects a disease-related region in the medical image data, the disease-related region being a region where a predetermined finding that may be related to the predetermined disease appears; generates feature amounts corresponding to the medical image data by using the plurality of first inference results acquired by the first inference and results of the detection of the disease-related region; and performs second inference about the predetermined disease to generate a second inference result corresponding to the medical image data by using the feature amounts as input.

### BACKGROUND

### Field of the Technology

The present disclosure relates to an information processing device and an information processing method.

### Description of the Related Art

A technology that infers a predetermined disease using medical image data as input is known. Japanese Patent Laid-Open No. 2020-188872 describes a technology that divides a medical image into a plurality of regions and infers whether the medical image contains a predetermined disease based on an estimation result of information related to the predetermined disease in each of the regions.

The technology described in Japanese Patent Laid-Open No. 2020-188872 could experience reduced inference performance depending on how the disease appeared in the medical image.

### SUMMARY

One aspect of the present disclosure provides a technology that can improve inference performance regarding a predetermined disease for a medical image.

One aspect of the present disclosure includes an information processing device including a memory storing instructions, and at least one processor configured to execute the instructions to: acquire medical image data; perform first inference about a predetermined disease to generate a plurality of first inference results corresponding to the plurality of first sub-regions by using a plurality of first sub-regions included in the medical image data as input; detect a disease-related region in the medical image data, the disease-related region being a region where a predetermined finding that may be related to the predetermined disease appears; generate feature amounts corresponding to the medical image data by using the plurality of first inference results acquired by the first inference and results of the detection of the disease-related region; and perform second inference about the predetermined disease to generate a second inference result corresponding to the medical image data by using the feature amounts as input.

Features of the present disclosure will become apparent from the following description of embodiments with reference to the attached drawings. The following description of embodiments is described by way of example.

### BRIEF DESCRIPTION OF THE DRAWINGS

FIG. 1 is a diagram illustrating an example of a functional configuration of an information processing device.

FIG. 2 is a diagram illustrating an example of a hardware configuration of the information processing device.

FIG. 3 is a flowchart illustrating an example of processing performed by the information processing device in a first embodiment.

FIG. 4 is a schematic diagram of the processing performed by the information processing device in the first embodiment.

FIG. 5 is a diagram illustrating an example of processing in a second embodiment.

FIG. 6 is a diagram illustrating an example of processing on a slice image in the second embodiment.

FIG. 7 is a diagram illustrating an example of feature amount generation in the second embodiment.

### DESCRIPTION OF THE EMBODIMENTS

Hereinafter, examples of embodiments of an information processing device, an information processing method, and a program for acquiring information about a predetermined disease from a medical image will be described with reference to the drawings. To acquire information about a predetermined disease, inference using machine learning models is used.

### First Embodiment

An information processing device in the first embodiment performs a first inference about a predetermined disease using each of a plurality of first sub-regions included in acquired medical image data as input, and acquires a plurality of first inference results corresponding to the plurality of first sub-regions, respectively. The information processing device also detects a region in the medical image data where a predetermined finding that may be related to the predetermined disease appears (referred to as a "disease-related region"). ‎ The information processing device then generates feature amounts corresponding to the medical image data using the plurality of first inference results and the detection results of the disease-related region, performs a second inference about the predetermined disease using the generated feature amounts as input, and acquires a second inference result corresponding to the medical image data.

In the present embodiment, as an example, a three-dimensional CT (Computed Tomography) image, more specifically a three-dimensional chest X-ray CT image, is used as medical image data, and the predetermined disease of interest is interstitial lung disease. In the first inference, an inference is made for each of the individual images (sub-regions) included in a three-dimensional chest X-ray CT image as to whether or not it contains a feature amount related to the predetermined disease (i.e., whether or not it contains an image feature of interstitial lung disease). In the second inference, a more detailed inference about the predetermined disease than in the first inference (e.g., an inference of the type of interstitial lung disease) is made. Of course, the target of interest is not limited to above, and they are merely examples for explaining the configuration and processing of the information processing device.

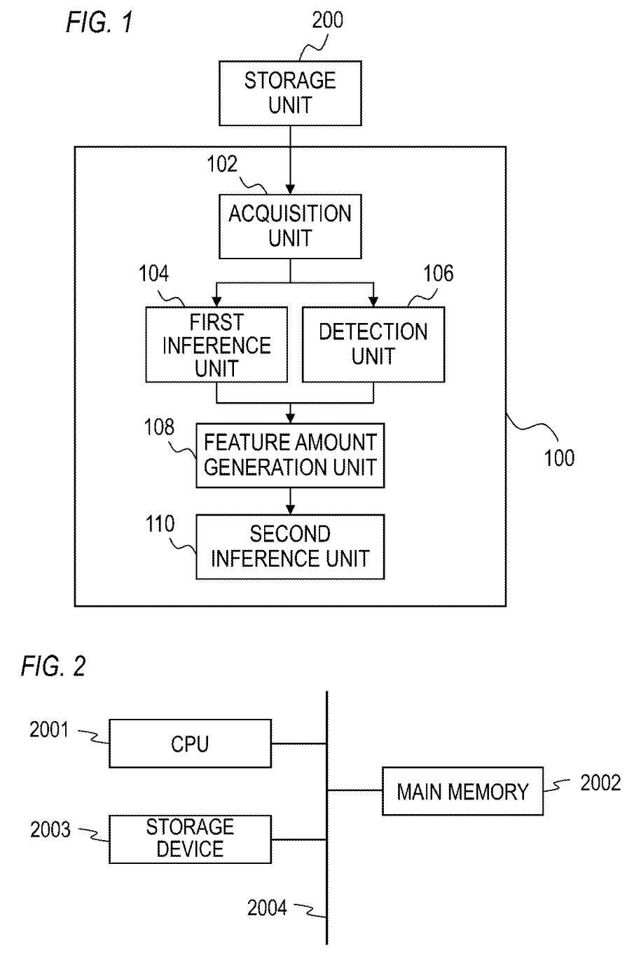

FIG. 1 is a diagram illustrating an example of a functional configuration of the information processing device 100 in the first embodiment. The information processing device 100 in the present embodiment is connected to a storage unit 200 that stores medical image data. Note that the storage unit 200 may store information other than medical image data. The storage unit 200 may also be a system such as a picture archiving and communication system (PACS) to provide medical image data in response to a request from the information processing device 100. The information processing device 100 may also be configured to connect to a medical imaging device (modality) (not illustrated) to directly acquire medical image data captured by the medical imaging device.

The information processing device 100 includes an acquisition unit 102, a first inference unit 104, a detection unit 106, a feature amount generation unit 108, and a second inference unit 110. The acquisition unit 102 makes a request to the storage unit 200 to acquire medical image data. The first inference unit 104 performs a first inference about a predetermined disease for each of a plurality of first sub-regions included in the medical image data. The detection unit 106 detects a disease-related region in the medical image data. For example, the detection unit 106 acquires one or more second sub-regions corresponding to the first sub-regions from the medical image data, and detects whether or not a disease-related region is included in each of second sub-regions. As used herein, a first sub-region and a second sub-region "correspond" to each other means, for example, that the first sub-region and the second sub-region overlap at least partially. The feature amount generation unit 108 generates feature amounts corresponding to the medical image data using the first inference results and the detection results of the disease-related region. This "feature amount" may be, for example, an index designed to indicate that image features of both a "predetermined disease" and a "predetermined finding" appear. The second inference unit 110 performs a second inference about the predetermined disease using the generated feature amounts as input, and acquires a second inference result corresponding to the medical image data.

At least some of the units of the information processing device 100 illustrated in FIG. 1 may be implemented as independent devices or circuits such as ASICs or FPGAs. The functions of each unit of the information processing device 100 may be implemented by information processing using a software program(s). Furthermore, some of the functions or processing of the information processing device 100 may be substituted by an external server through distributed computing or cloud computing. In the present embodiment, it is assumed that the functions illustrated in FIG. 1 are implemented by information processing using a software program(s).

FIG. 2 is a diagram illustrating an example of a hardware configuration of the information processing device 100. The information processing device 100 is configured as a computer including, as its main components, a CPU (processor) 2001, a main memory 2002, and a storage device 2003. The CPU 2001 mainly controls the operation of each component of the information processing device 100. The main memory 2002 stores control programs executed by the CPU 2001 and provides a work area for the CPU 2001 to execute the programs. The storage device 2003 is a device that non-temporarily stores an operating system (OS), device drivers for peripheral devices, programs for performing processing described below, and the like. The storage device 2003 is configured, for example, with a magnetic disk (HDD) or a solid-state drive (SSD). The CPU 2001 executes programs stored in the main memory 2002 and the storage device 2003 to implement the functions (software) of the information processing device 100 illustrated in FIG. 1 and processing in flowcharts described below. The above components are communicably connected to one another via a common bus 2004. Note that the information processing device 100 may include a GPU, a display device, an input device, an output device, a communication I/F, and the like.

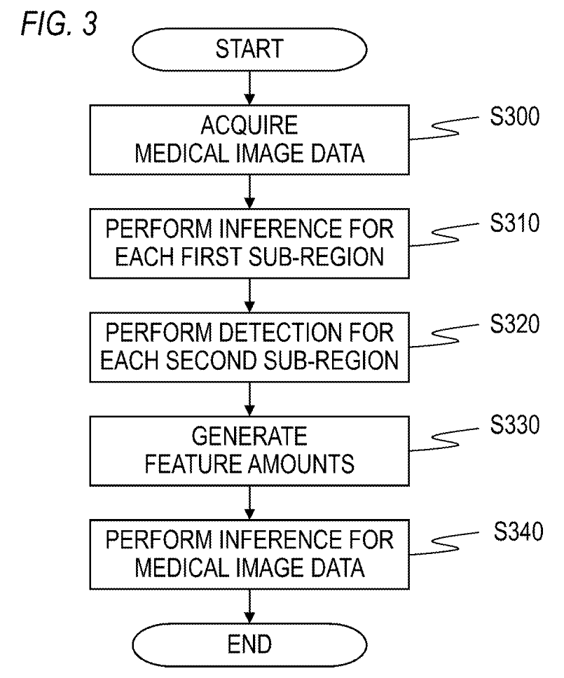

Next, the overall processing performed by the information processing device 100 will be described with reference to a flowchart of FIG. 3. FIG. 3 is a flowchart illustrating an example of processing performed by the information processing device 100. In the present embodiment, the CPU 2001 executes a program stored in the main memory 2002 or the storage device 2003 to implement the processing illustrated in FIG. 3.

In step S300, the acquisition unit 102 makes a request to the storage unit 200 to acquire a three-dimensional chest X-ray CT image as medical image data. The three-dimensional chest X-ray CT image is composed of a plurality of two-dimensional slice images of body axial cross sections.

In step S310, the first inference unit 104 performs a first inference about a predetermined disease for each of a plurality of first sub-regions included in the medical image data acquired in step S300. The first inference unit 104 of the present embodiment uses a two-dimensional slice image of a body axial section as a first sub-region, and performs an inference as to whether or not the two-dimensional slice image contains an image feature indicative of interstitial lung disease.

In response to receiving a first sub-region (two-dimensional slice image) as input, the first inference unit 104 performs the first inference using a machine learning model configured to output the presence or absence of the predetermined disease. A known algorithm (e.g., support vector machine, Random Forest, deep learning, etc.) can be used as the machine learning model. The machine learning model is pre-trained by supervised learning using a large amount of teacher data, and parameter tuning may be performed as necessary. A group of samples of two-dimensional slice images of chest X-ray CT images acquired from many subjects is used as the teacher data. Each sample is assigned a label, as ground truth data, indicating whether or not it contains any image feature indicative of the predetermined disease (in the present embodiment, "interstitial lung disease"). The ground truth data is labeled (annotated) by a user, such as a doctor.

In step S320, the detection unit 106 acquires a plurality of second sub-regions included in the medical image data acquired in step S300, and detects a disease-related region for each of second sub-regions. The second sub-regions used for detection in step S320 are set to correspond to the first sub-regions used for the first inference in step S310. In the present embodiment, the first sub-region and the second sub-region are the same. However, the regions are not limited to this example, and some of the first sub-regions may be set as the second sub-regions, or a region including a first sub-region may be set as a second sub-region. Alternatively, the first sub-region and the second sub-region may be set so that they partially overlap. As used herein, the "region" may be a two-dimensional region or a three-dimensional region. The disease-related region to be detected by the detection unit 106 is a region in which a predetermined finding that may be associated with the predetermined disease appears. In the present embodiment, the predetermined disease is interstitial lung disease, and therefore honeycomb lung is treated as the predetermined finding. Honeycomb lung refers to a finding in which alveoli change into cysts and cluster together to form a honeycomb pattern due to pulmonary fibrosis, and is an important clue when diagnosing interstitial lung disease. The detection unit 106 of the present embodiment detects honeycomb lung from two-dimensional slice images using a known method (e.g., a deep learning method described in "Lung pattern classification for interstitial lung diseases using a deep convolutional neural network", Anthimopoulos, M. et al., IEEE TRANSACTIONS ON MEDICAL IMAGING, 35(5), 1207-16, 2016).

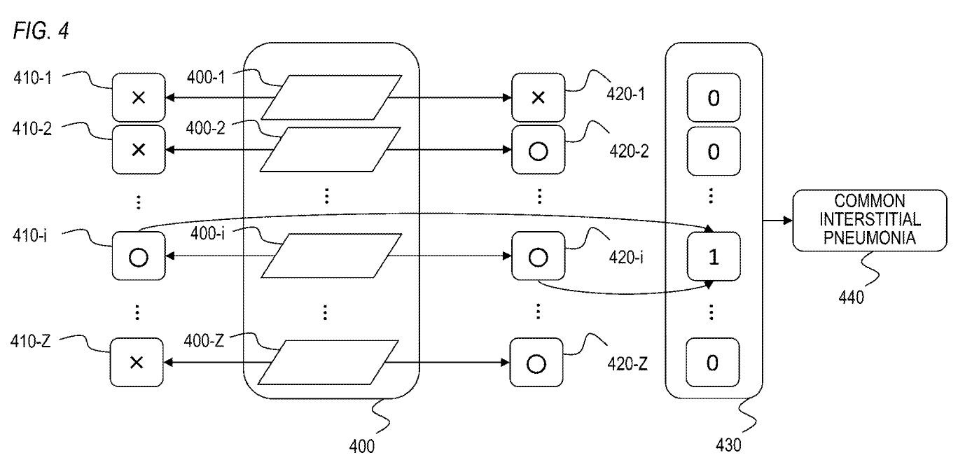

In step S330, the feature amount generation unit 108 generates feature amounts corresponding to the medical image data using the first inference results acquired in step S310 and the detection results of the disease-related region acquired in step S320. A specific example of generating feature amounts will be described later with reference to FIG. 4.

In step S340, the second inference unit 110 performs the second inference about the predetermined disease (interstitial lung disease) using the feature amounts generated in step S330 as input, and acquires a second inference result corresponding to the medical image data. The first inference and the second inference have in common that they are both inferences about the predetermined disease, but the first inference and the second inference have different given propositions (problems to be solved). For example, the second inference may be processing that performs a more detailed inference about the predetermined disease than the first inference. In other words, the first inference may be an inference in a higher-level class (an inference that solves a higher-level proposition) than the second inference. In the present embodiment, the first inference performs classification as to whether or not the two-dimensional slice image contains image feature related to interstitial lung disease, and the second inference classifies the type of interstitial lung disease. More specifically, in the second inference, classification is performed as to whether common interstitial pneumonia, nonspecific interstitial pneumonia, or something else appears.

In response to receiving the feature amounts generated by the feature amount generation unit 108 as input, the second inference unit 110 performs the second inference using a machine learning model configured to output the type of the predetermined disease. A known algorithm (e.g., support vector machine, Random Forest, deep learning, etc.) can be used as the machine learning model. The machine learning model is pre-trained by supervised learning using a large amount of teacher data, and parameter tuning may be performed as necessary. A group of samples of feature amounts acquired by performing the processing of steps S310 to S330 on three-dimensional chest X-ray CT images acquired from a plurality of subjects is used as the teacher data. Each sample is assigned a label, as ground truth data, indicating the type of the predetermined disease (in the present embodiment, "interstitial lung disease", or "something else"). The ground truth data is labeled (annotated) by a user, such as a doctor. Alternatively, the ground truth data may be acquired on the basis of definitive diagnosis information recorded in an electronic medical record or the like.

FIG. 4 is a schematic diagram of the processing performed by the information processing device 100 in the present embodiment. Medical image data 400 is a three-dimensional CT image that is composed of Z consecutive two-dimensional slice images 400-1 to 400-Z of body axial cross sections (Z is an integer greater than 1, e.g., approximately tens to hundreds). In the present embodiment, one two-dimensional slice image is a first sub-region on which the first inference is to be performed and also a second sub-region in which a disease-related region is to be detected. Hereinafter, the i-th two-dimensional slice image will be represented as 400-i (i = 1, ..., Z).

A first inference result 410-i is an inference result obtained by the first inference unit 104 performing the first inference about interstitial lung disease using the two-dimensional slice image 400-i, which is a first sub-region, as input. In FIG. 4, the first inference result that an image feature of interstitial lung disease are contained in the two-dimensional slice image is indicated by "○", and the first inference result that any image feature of interstitial lung disease is not contained is indicated by "×".

A detection result 420-i indicates the detection result obtained by the detection unit 106 detecting honeycomb lung (disease-related region) from the two-dimensional slice image 400-i, which is a second sub-region. As described above, in the present embodiment, the first sub-region and the second sub-region are the same region. Therefore, when the two-dimensional slice image 400-i is input to the first inference unit 104 as the first sub-region and the first inference is then performed, the second sub-region to be detected by the detection unit 106 is the same two-dimensional slice image 400-i as the first sub-region. Here, first, for the pixels constituting the two-dimensional slice image 400-i, which is in turn the second sub-region, information of a binary value of 0/1 indicative of whether or not each pixel indicates honeycomb lung is obtained. If there is even one pixel indicating honeycomb lung, a detection result is obtained that honeycomb lung has been detected in the slice. On the other hand, if there is not even one pixel indicating honeycomb lung, a detection result is obtained that honeycomb lung has not been detected in the slice. Of course, this is just one example, and a detection result may be obtained using other information and determination methods. In FIG. 4, the detection result obtained that honeycomb lung is included in the two-dimensional slice image is indicated by "○", and the detection result obtained that honeycomb lung is not included is indicated by "×".

A feature amount 430 is a feature amount generated by the feature amount generation unit 108 using the first inference results 410-1 to 410-Z and the detection results 420-1 to 420-Z. In the present embodiment, the feature amount 430 is a Z-dimensional vector that is composed of Z elements, each of which corresponds to the two-dimensional slice image 400-i (i.e., the first sub-region and the second sub-region). When both the first inference result 410-i and the detection result 420-i are positive ("○"), that is, when the features of interstitial lung disease are observed and findings of honeycomb lung are also observed, such information is considered to be a clue for the second inference to be performed subsequently, and the corresponding element is set to a value of "1". When either or both of the first inference result 410-i and the detection result 420-i are negative ("×"), such information is considered not to be a clue for the second inference to be performed subsequently, and the corresponding element is set to a value of "0". In other words, when any feature of interstitial lung disease is observed but findings of honeycomb lung are not, when findings of honeycomb lung are observed but any feature of interstitial lung disease is not, and when neither any feature of interstitial lung disease nor findings of honeycomb lung are observed, the corresponding element is set to a value of "0".

Whether a slice exhibits any feature of interstitial lung disease cannot be determined solely by the presence or absence of honeycomb lung. However, honeycomb lung is detected based on whether it has any feature of honeycomb lung, it can also be detected in a slice (second sub-region) that is determined to exhibit no features of interstitial lung disease as a whole. Therefore, if a feature amount is generated using only the detection results of honeycomb lung, the generated feature amount will contain information that is not actually suitable for determining the type of interstitial lung disease. Since a doctor focuses on honeycomb lung in slices that exhibit any feature of interstitial lung disease, the inference performance can be improved by treating honeycomb lung information detected in a slice that exhibits no features of interstitial lung disease as noise and not using it to generate a feature amount.

The second inference unit 110 performs the second inference using the feature amount 430 generated in this way as input, and obtains a second inference result 440 (the classification result of "common interstitial pneumonia" in FIG. 4).

In the present embodiment, a feature amount is generated using a first inference result indicating whether or not a first sub-region contains any feature of the predetermined disease (interstitial lung disease) and a detection result of the disease-related region (honeycomb lung) in a second sub-region corresponding to the first sub-region. In this generation, using a correspondence (one-to-one correspondence in the present embodiment) between the first sub-region and the second sub-region (the same region in the present embodiment), only the sub-region for which both the first inference result and the detection result are positive is used as information that is considered to be a clue for the second inference. This makes it possible to eliminate in advance information that may become noise during the second inference, thereby improving the inference performance of the second inference.

The above-described configuration and processing are merely an example for explaining the information processing device. For example, the first sub-region and the second sub-region do not have to be exactly the same. An example of a case where they are not exactly the same will be described in a second embodiment.

### Second Embodiment

A configuration of the second embodiment will be described below, but a description of the same configuration as the first embodiment will be omitted. An information processing device 100 in the second embodiment differs from the information processing device 100 in the first embodiment mainly in the following respects.
The first sub-region and the second sub-region are not the same;
The detection unit 106 detects the disease-related region using two detection methods; and
The first inference unit 104 acquires, as a first inference result, a likelihood of the first sub-region containing any image feature related to a predetermined disease.

In the following, as in the first embodiment, a three-dimensional chest X-ray CT image is used as medical image data, and the first inference to be performed is an inference about interstitial lung disease, and the second inference to be performed is an inference about the type of interstitial lung disease. Of course, the target of interest is not limited to above, and they are merely examples for explaining processes of the information processing device.

The functional configuration, hardware, and processing procedure of the information processing device 100 in the second embodiment are the same as those in the first embodiment. Only the differences from the first embodiment will be described below based on the flowchart of FIG. 3.

The processing of step S300 referred to herein is similar to the processing of step S300 in the first embodiment.

In step S310, the first inference unit 104 performs the first inference about the predetermined disease for a first sub-region included in the medical image data acquired in step S300. The first inference unit 104 of the present embodiment uses a two-dimensional slice image of a body axial cross section as the first sub-region, and performs an inference of the likelihood of the two-dimensional slice image containing any image feature of interstitial lung disease.

In response to receiving a first sub-region (two-dimensional slice image) as input, the first inference unit 104 performs the first inference using a machine learning model configured to output a likelihood of the first sub-region containing any image feature related to the predetermined disease. A known algorithm (e.g., support vector machine, Random Forest, deep learning, etc.) can be used as the machine learning model. The machine learning model is pre-trained by supervised learning using a large amount of teacher data, and parameter tuning may be performed as necessary. A group of samples of two-dimensional slice images of chest X-ray CT images acquired from many subjects is used as the teacher data. Each sample is assigned a label, as ground truth data, indicating a likelihood of the two-dimensional slice image containing any image feature related to the predetermined disease (in the present embodiment, "interstitial lung disease"). The ground truth data is labeled (annotated) by, for example, a doctor.

In step S320, the detection unit 106 acquires a plurality of second sub-regions included in the medical image data acquired in step S300, and detects a disease-related region for each second sub-region. The detection unit 106 of the present embodiment includes a first detection unit and a second detection unit that detect the disease-related region using different detection methods (detection algorithms; models), and detects the disease-related region in the same second sub-region using the two types of detection methods.

In the present embodiment, the second sub-region is a region obtained by dividing the first sub-region. In other words, the second sub-region is included in the first sub-region, and a plurality of second sub-regions are linked to the first sub-region. This is an example where the first sub-region and the second sub-region overlap at least partially. For example, when the first sub-region is divided into four, four second sub-regions corresponding to the first sub-region are obtained. For each of the four second sub-regions, detection of the disease-related region is performed using a first detection method by the first detection unit, and detection of the disease-related region is performed using a second detection method by the second detection unit. As a result, eight detection results are obtained for one first sub-region.

The first detection unit and the second detection unit, which can use known methods for detecting honeycomb lung, use different detection methods. For example, when using the deep learning method described in "Lung pattern classification for interstitial lung diseases using a deep convolutional neural network", the first detection unit and the second detection unit may have different model configurations, or may use models with different inference performance by training using different pieces of teacher data. Furthermore, the first detection unit and the second detection unit may use different machine learning algorithms.

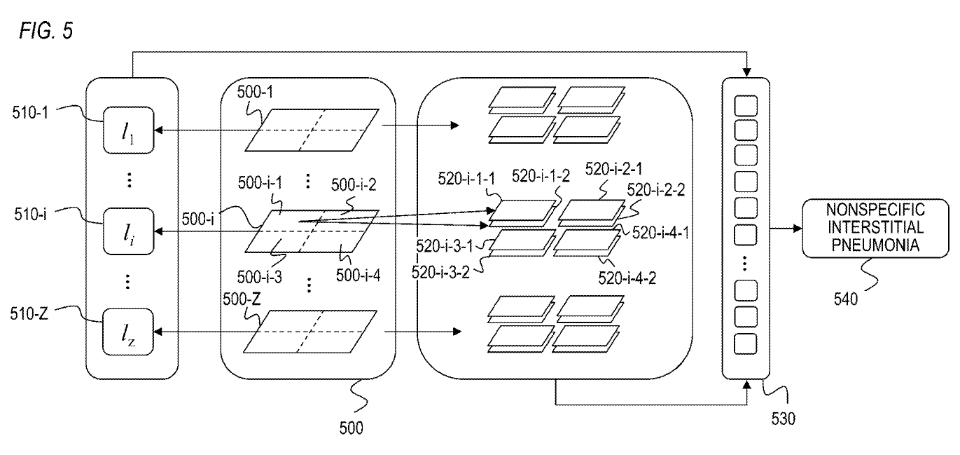

In step S330, the feature amount generation unit 108 generates a feature amount using the first inference results obtained in step S310 and the detection results of the disease-related region by the first detection unit and the detection results of the disease-related region by the second detection unit, which are obtained in step S320. A specific example of generating a feature amount will be described later with reference to FIGS. 5-7.

The processing of step S340 referred to herein is similar to the processing of step S340 in the first embodiment.

FIG. 5 is a schematic diagram of processing performed by the information processing device 100 in the present embodiment. Medical image data 500 is a three-dimensional CT image that is composed of Z consecutive two-dimensional slice images 500-1 to 500-Z of body axial cross sections. In the present embodiment, one two-dimensional slice image is a first sub-region on which the first inference is to be performed, and a division of the two-dimensional slice image is a second sub-region in which a disease-related region is to be detected. Hereinafter, the i-th two-dimensional slice image will be represented as 500-i (i = 1, ..., Z). Furthermore, a region obtained by dividing the two-dimensional slice image 500-i (i.e., a second sub-region) will be represented as 500-i-j (j = 1, ...). In the example of FIG. 5, the two-dimensional slice image 500-i is divided into four equal-sized 2×2 regions, resulting in second sub-regions 500-i-1 to 500-i-4. The division method and/or the number of divisions may be the same for all two-dimensional slice images or may be different for all two-dimensional slice images. In the present embodiment, all two-dimensional slice images are divided using the same division method and number of divisions to set second sub-regions.

The first inference result 510-i is an inference result obtained by the first inference unit 104 performing the first inference about interstitial lung disease using the two-dimensional slice image 500-i, which is a first sub-region, as input. The first inference result 510-i is a likelihood of the two-dimensional slice image 500-i containing any image feature of interstitial lung disease. The likelihood takes a value between 0 and 1 (hereinafter referred to as li).

A first detection result 520-i-j-1 is a detection result obtained by the detection unit 106 detecting honeycomb lung (disease-related region) from a second sub-region 500-i-j using a first detection method. A second detection result 520-i-j-2 is a detection result obtained by the detection unit 106 detecting honeycomb lung (disease-related region) from the second sub-region 500-i-j using a second detection method.

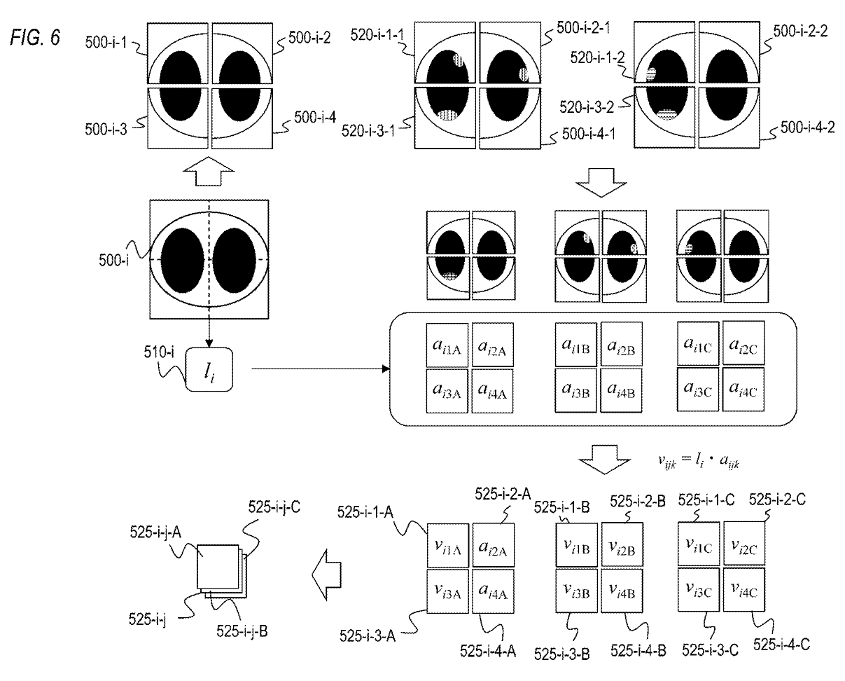

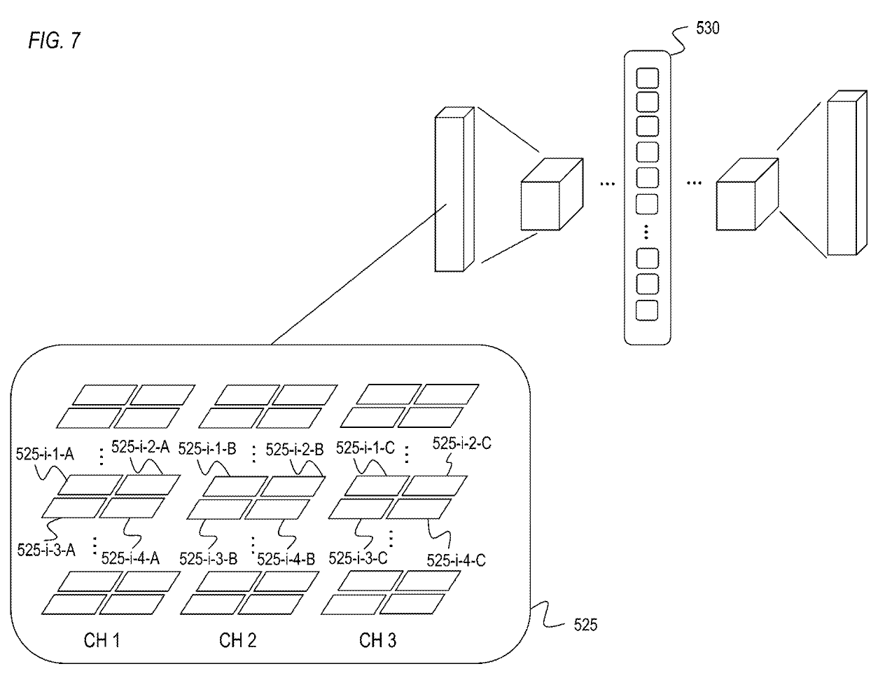

A feature amount 530 is a feature amount generated by the feature amount generation unit 108 using the first inference results, the first detection results, and the second detection results. This will be described in detail with reference to FIGS. 6 and 7.

FIG. 6 is a schematic diagram of processing for the two-dimensional slice image 500-i. In the present embodiment, pseudo channels 525-i-j-A to 525-i-j-C are generated using first detection results 520-i-j-1, second detection results 520-i-j-2, and the first inference results 510-i. Then, three-channel pseudo-pixels 525-i-j having these channel values are generated. Note that, in the present embodiment, the first and second detection results are information indicating whether each of the pixels constituting a second sub-region is indicative of honeycomb lung using a binary value of 0/1.

First, a volume of honeycomb lung detected in both a first detection result 520-i-j-1 and a second detection result 520-i-j-2 is acquired. This volume can be determined by counting pixels constituting the second sub-region 500-i-j, each of which is indicated as honeycomb lung in both the first detection result 520-i-j-1 and the second detection result 520-i-j-2. In the present embodiment, since the second sub-region is a two-dimensional region obtained by dividing the two-dimensional slice image 500-i, the total of the pixels represents its area, and the area can be considered as a volume with a depth of 1. Hereinafter, this area (volume) will be referred to as aijA. Similarly, pixels constituting the second sub-region 500-i-j, each of which is indicated as honeycomb lung in the first detection result 520-i-j-1 but not as honeycomb lung in the second detection result 520-i-j-2, are counted to obtain an area aijB. Furthermore, pixels constituting the second sub-region 500-i-j, each of which is indicated as honeycomb lung in the second detection result 520-i-j-2 but is not indicated as honeycomb lung in the first detection result 520-i-j-1, are counted to obtain an area aijC. In the present embodiment, the area aijA is an example of first information indicating that the disease-related region has been detected by both the first detection method and the second detection method. The areas aijB and aijC are each an example of second information indicating that the disease-related region has been detected by only one of the first detection method and the second detection method.

Next, the areas aijA to aijC obtained here are each multiplied by the first inference result 510-i for the first sub-region (two-dimensional slice image) 500-i linked to the second sub-region 500-i-j to obtain values vijA to vijC. These values vijA to vijC are scores obtained by weighting the detection results for the second sub-regions using the likelihood of the first sub-region, utilizing the correspondence between the first sub-region and the second sub-region. Then, pseudo channels 525-i-j-A to 525-i-j-C are generated, each having these scores as its channel value. Then, a pseudo-pixel 525-i-j having the three channel values are generated. In this way, by using first information indicating successful detection by both the detection methods and second information indicating that successful detection only by one of the methods, it is possible to take into account detection results that do not depend on either detection method and detection results that are specific to one detection method. Since the first inference result is a likelihood that the slice image exhibits any feature of interstitial lung disease, the pseudo-pixels represent the detection results of disease-related regions weighted by the likelihood that the slice image exhibits the feature.

This processing is applied to all two-dimensional images (i.e., all first sub-regions and second sub-regions), and a pseudo image 525 as illustrated in FIG. 7 is generated from the resulting pseudo pixels. In the present embodiment, since each of the Z two-dimensional slice images is divided into 2×2, the pseudo image 525 ultimately generated has a height of 2, a width of 2, a depth of Z, and three channels.

In the present embodiment, this pseudo image 525 is input to a trained convolutional autoencoder, and the value of an intermediate layer is acquired as the feature amount 530 (i.e., the feature amount is generated using the convolutional autoencoder). This is an example of generating a feature amount using convolution processing. The convolutional autoencoder is a well-known technique that performs convolution processing on an image as input, and then performs deconvolution to restore the input image. By extracting the intermediate layer of the convolutional autoencoder, it is possible to generate a feature amount that contains information on the relationship between adjacent images in each direction and channel.

The second inference unit 110 performs the second inference using the feature amount 530 generated in this way as input, and obtains the second inference result 540 (nonspecific interstitial pneumonia herein).

In the present embodiment, for first sub-regions included in medical image data, a feature amount is generated from first inference results, which are likelihoods that the first sub-regions contain any image feature of interstitial lung disease, and detection results of the disease-related region for second sub-regions corresponding to the first sub-regions. In this case, the disease-related region is detected using two types of detection methods, and first information indicating that the region disease-related region has been detected by both the detection methods and second information indicating that the disease-related region has been detected by only one of the detection methods are used. This makes it possible to generate a feature amount taking into account detection results that do not depend on either detection method and detection results that are specific to one detection method, thereby improving the inference performance.

The feature amount generation unit 108 generates a feature amount by performing convolution processing on pseudo images generated from a plurality of first inference results acquired from a plurality of first sub-regions and from a plurality of detection results acquired from a plurality of second regions. In this way, a feature amount that includes the relationship between adjacent images in each direction is generated, making it possible to generate the feature amount taking into account the positions in the detection results, thereby improving the inference performance.

Furthermore, by utilizing the correspondence between the first sub-region and the second sub-region and weighting the detection result for the second sub-region using the likelihood of the first sub-region, the influence of information that becomes noise during the second inference can be reduced, thereby improving the inference performance.

### Modification Example 1

In the first and second embodiments, two-dimensional slice images and/or divided images of two-dimensional slice images, i.e., two-dimensional images, are used as the first sub-regions and the second sub-regions. However, they do not have to be two-dimensional images as long as they are sub-regions of medical image data. For example, they may each be a part of a three-dimensional image such as a volume of interest (VOI), or a three-dimensional image that is composed of a plurality of two-dimensional slice images. As in the first sub-region being a VOI and the second sub-region being an orthogonal two-dimensional cross section of the VOI, the first sub-region and the second sub-region may differ in the number of dimensions. Furthermore, the second sub-region only needs to at least partially overlap with the first sub-region, and does not have to be the same region as the first sub-region or a divided region. For example, the second sub-region may be larger than the first sub-region, or the first sub-region and the second sub-region may have non-overlapping regions.

Furthermore, in the first and second embodiments, a three-dimensional chest X-ray CT image that is composed of a plurality of two-dimensional images of body axial cross sections is acquired as medical image data. However, the image is not limited to this, and may be composed of a plurality of two-dimensional slice images of any cross sections, such as coronal sections or sagittal sections. Instead of being composed of a plurality of two-dimensional slice images, it may be a three-dimensional image that is composed of voxel data. Furthermore, the medical image data may be a four-dimensional image having data in time domain, and is not limited by the number of dimensions.

### Modification Example 2

In the first and second embodiments, the first inference about a predetermined disease is performed for a first sub-region in step S310, and then a disease-related region related to the predetermined disease is detected in a second sub-region in step S320. However, the processing does not necessarily have to be in this order. For example, the first inference may be performed after detection of the disease-related region. The detection of the disease-related region and the first inference may be performed in parallel.

Although the detection of the disease-related region is performed regardless of the first inference results, the detection of the disease-related region may be performed depending on the first inference results. Specifically, if a first inference result satisfies a predetermined condition, the detection of the disease-related region may be performed, and if the first inference result does not satisfy the predetermined condition, the detection of the disease-related region may not be performed. For example, in the example of the first embodiment, the detection may be performed only when the inference indicates "○" and in the example of the second embodiment, the detection may be performed only when the likelihood is equal to or greater than a specified value. Conversely, if the detection of the disease-related region is performed first, the first inference may be performed according to the detection results of the disease-related region. Specifically, if a detection result of the disease-related region satisfies a predetermined condition, the first inference may be performed, and if the detection result of the disease-related region does not satisfy the predetermined condition, the first inference may not be performed. In this way, the overall processing time can be reduced.

### Modification Example 3

In the second embodiment, the processing is performed on second sub-regions obtained by dividing a first sub-region in step S320. However, it is not necessary to perform the processing after dividing the first sub-region. For example, detection processing of a disease-related region related to a predetermined disease may be performed on a first sub-region, and the detection results for second sub-regions may be used depending on the positions in the detection results within the first sub-region. This makes it possible to reduce the adverse effects of division, such as a disease-related region existing on a division boundary line that was detectable before division but becomes undetectable after division.

### Modification Example 4

In the second embodiment, the volume (area) of the disease-related region is used to generate a feature amount in step S330, but the volume (area) is not necessarily used. For example, the number of closed regions (i.e., the number of disease-related regions) may be calculated on the basis of information indicating whether each of the pixels constituting a second sub-region indicative of honeycomb lung using a binary value of 0/1, and the feature amount may then be generated. This is particularly useful when the second inference is better based on the number of occurrences of disease-related regions rather than their extent.

One aspect of the present disclosure can improve the inference performance about a predetermined disease for a medical image.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a 'non-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)^{TM}), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-082589, filed May 16, 2025, which is hereby incorporated by reference herein in its entirety.

What is claimed is:

1. An information processing device comprising:
a memory storing instructions, and
at least one processor configured to execute the instructions to:
acquire medical image data;
perform first inference about a predetermined disease to generate a plurality of first inference results corresponding to the plurality of first sub-regions by using a plurality of first sub-regions included in the medical image data as input;
detect a disease-related region in the medical image data, the disease-related region being a region where a predetermined finding that may be related to the predetermined disease appears;
generate feature amounts corresponding to the medical image data by using the plurality of first inference results acquired by the first inference and results of the detection of the disease-related region; and
perform second inference about the predetermined disease to generate a second inference result corresponding to the medical image data by using the feature amounts as input.

2. The information processing device according to claim 1, wherein the at least one processor is configured to detect the disease-related region in a region corresponding to each of the plurality of first sub-regions.

3. The information processing device according to claim 1, wherein the plurality of first sub-regions are a plurality of slice images.

4. The information processing device according to claim 1, wherein each of the plurality of first inference result is information indicating whether or not each of the plurality of first sub-region contains any feature related to the predetermined disease.

5. The information processing device according to claim 1, wherein each of the plurality of first inference result is a likelihood of each of the plurality of first sub-region containing any feature related to the predetermined disease.

6. The information processing device according to claim 1, wherein the at least one processor is configured to generate the feature amounts by using a number or volume of the disease-related regions.

7. The information processing device according to claim 1, wherein the plurality of first inference results includes likelihood of each of the plurality of first sub-region containing any feature related to the predetermined disease, and
wherein the at least one processor is configured to generate the feature amounts corresponding to the medical image data by using the likelihood acquired as the plurality of first inference result and a number or volume of the disease-related regions. ‎

8. The information processing device according to claim 1, wherein the at least one processor is configured to (i) detect the disease-related region from the medical image data using a first detection method, and (ii) detect the disease-related region from the medical image data using a second detection method different from the first detection method, and
generate the feature amounts by using at least one of first information and second information, the first information indicating that the disease-related region has been detected by both the first detection method and the second detection method, the second information indicating that the disease-related region has been detected by only one of the first detection method and the second detection method.

9. The information processing device according to claim 1, wherein the at least one processor is configured to detect whether or not each of the plurality of second sub-regions, corresponding to each of the plurality of first sub-regions, contains the disease-related region.

10. The information processing device according to claim 9, wherein the at least one processor is configured to detect, in a case where one of the plurality of first inference results for one of the plurality of first sub-regions satisfies a predetermined condition, the disease-related region in a second sub-region corresponding to the one of the plurality of first sub-regions, and not to detect, in a case where the one of the plurality of first inference results for the one of the plurality of first sub-regions does not satisfy the predetermined condition, the disease-related region for the second sub-region corresponding to the one of the plurality of first sub-regions.

11. The information processing device according to claim 9, wherein the at least one processor is configured to perform, in a case where a detection result for the plurality of second sub-regions satisfies a predetermined condition, the first inference for a first sub-region corresponding to the one of the plurality of second sub-regions, and not to perform, in a case where the detection result for the plurality of second sub-regions does not satisfy the predetermined condition, the first inference for the first sub-region corresponding to the one of the plurality of second sub-regions.

12. The information processing device according to claim 9, wherein the at least one processor is configured to generate the feature amounts by performing convolution processing on a pseudo image, wherein the pseudo image is generated by using (i) the plurality of first inference results acquired from the plurality of first sub-regions, and (ii) the results of the detection of the disease-related region in the plurality of second sub-regions corresponding to the plurality of first sub-regions.

13. The information processing device according to claim 1, wherein the second inference is processing to perform a more detailed inference about the predetermined disease than the first inference.

14. The information processing device according to claim 1, wherein
the medical image data is a three-dimensional CT image that is composed of a plurality of two-dimensional slice images, and
the first sub-region is the two-dimensional slice image included in the three-dimensional CT image.

15. The information processing device according to claim 14, wherein
the predetermined disease is interstitial lung disease, and
the predetermined finding is honeycomb lung.

16. An information processing method executed by a processor, the information processing method comprising:
acquiring medical image data;
performing first inference about a predetermined disease to generate a plurality of first inference results corresponding to the plurality of first sub-regions by using a plurality of first sub-regions included in the medical image data as input;
detecting a disease-related region in the medical image data, the disease-related region being a region where a predetermined finding that may be related to the predetermined disease appears;
generating feature amounts corresponding to the medical image data by using the plurality of first inference results acquired by the first inference and results of the detection of the disease-related region; and
performing second inference about the predetermined disease to generate a second inference result corresponding to the medical image data by using the feature amounts as input.

17. A non-transitory computer readable medium that stores a program, wherein the program causes a computer to execute an information processing method comprising:
acquiring medical image data;
performing first inference about a predetermined disease to generate a plurality of first inference results corresponding to the plurality of first sub-regions by using a plurality of first sub-regions included in the medical image data as input;
detecting a disease-related region in the medical image data, the disease-related region being a region where a predetermined finding that may be related to the predetermined disease appears;
generating feature amounts corresponding to the medical image data by using the plurality of first inference results acquired by the first inference and results of the detection of the disease-related region; and
performing second inference about the predetermined disease to generate a second inference result corresponding to the medical image data by using the feature amounts as input.
